(19) United States
(12) Patent Application Publication
(10) Pub. No.: US 2019/0237173 A1
(43) Pub. Date: Aug. 1, 2019

(54) ACTION PLANNER SYSTEMS AND METHODS TO SIMULATE AND CREATE A RECOMMENDED ACTION PLAN FOR A PHYSICIAN AND A CARE TEAM, OPTIMIZED BY OUTCOME

(71) Applicant: ARMADAHEALTH LLC, Hunt Valley, MD (US)

(72) Inventors: Steven Edward Schaefer, Lutherville, MD (US); Bharath Sudharsan, Abingdon, MD (US); Calvin Christian Wilhide, III, Lutherville-Timonium, MD (US)

(21) Appl. No.: 16/380,065

(22) Filed: Apr. 10, 2019

Related U.S. Application Data
(60) Provisional application No. 62/655,346, filed on Apr. 10, 2018.

(51) Int. Cl. G16H 10/60; G16H 50/20; G16H 50/50; G16H 50/70

(52) U.S. Cl. CPC G16H 10/60; G16H 50/20; G16H 40/20; G16H 50/70; G16H 50/50

(57) ABSTRACT

Systems and methods for action plan optimization for multiple stakeholders. A user interface receives data comprising at least one of patient, physician and care team characteristics. A scenario manager organizes the received data into multiple categories of information. A scenario simulator: identifies possible scenarios and combinations of interactions between the patient, physician and care team with a respect to a predefined healthcare outcome, based on the multiple categories of information, and defines a set of potential outcomes among the possible scenarios and combinations via simulation according to statistical algorithms, based on the predefined healthcare outcome. An analysis engine determines an optimized clinical pathway based on the set of potential outcomes, according to further statistical algorithms. A role-based planner determines an electronic action plan based on the optimized clinical pathway. The electronic action plan identifies roles and actions for each of the patient, the physician and the care team.

### TECHNICAL FIELD

The present disclosure relates generally to patient healthcare management techniques and, in particular, to systems and methods of simulating and generated an electronic recommended action plan for a physician and a care team, optimized for one or more outcomes.

### BACKGROUND

Patient healthcare management systems are known. Conventional solutions focus on using clinical workflows and behavioral change interventions to optimize clinical and behavioral outcomes of a patient's healthcare. These solutions, however, do not focus on social, behavioral and psychosocial attributes of both the patient and the care team, including the physician. Instead, conventional solutions focus solely on the patient. Yet further, these solutions just consider clinical factors and social determinants. Current solutions also do not predict a patient's healthcare progress and its effect on outcomes over a healthcare process. Yet further conventional solutions are not adaptive and do not consider any course-corrections or recommendations based on a patient's progress over time.

Yet further, conventional management systems do not consider factors associated with the physician and the care team over a healthcare process as well, holistically, and in turn their effect on one or more patient-specific outcomes. Moreover, conventional management systems do not consider optimizing outcome around quality of life outcomes for the care team and the physician (e.g., in addition to any quality of life considerations of the patient). Further, management systems typically focus on a standard list of clinical and social determinants. Conventional systems do not consider various irrational personality-based and family-specific determinants (e.g., for the patient, physician and care team) that could influence outcomes of the patient's healthcare progress.

Conventional systems also do not update any simulations for each new patient or for a given cohort of patients. Rather, conventional systems just take a standard assumed cohort and simulated clinical and region of interest (ROI) outcomes; and only focus on models around the ROI and clinical outcomes, not behavioral and qualitative outcomes like quality of life.

### SUMMARY

Aspects of the present disclosure relate to systems, methods and non-transitory computer readable mediums for creating an optimized action plan for multiple stakeholders. An optimized action planner system includes a user interface, a scenario manager, a scenario simulator, an analysis engine and a role-based planner. The user interface is configured to receive data comprising at least one of characteristics of a patient, a physician and one or more other treatment personnel defining a care team. The scenario manager is configured to organize the received data into multiple categories of information. The scenario simulator is configured to identify a plurality of possible scenarios and possible combinations of interactions between the patient, the physician and the care team with a respect to a predefined healthcare outcome, based at least in part on the multiple categories of information. The scenario manager is further configured to define a set of potential outcomes among the plurality of possible scenarios and possible combinations via simulation according to one or more statistical algorithms, based on the predefined healthcare outcome. The analysis engine is configured to determine an optimized clinical pathway based on the set of potential outcomes, according to one or more further statistical algorithms. The role-based planner is configured to determine an electronic action plan based on the optimized clinical pathway. The electronic action plan identifies one or more roles and one or more actions for each of the patient, the physician and the care team.

### BRIEF DESCRIPTION OF DRAWINGS

FIG. 1 is a functional block diagram of an example optimized action planner system, according to an aspect of the present disclosure.

FIG. 2 is a functional block diagram of an example data warehouse associated with the system shown in FIG. 1, according to an aspect of the present disclosure.

FIG. 3 is a functional block diagram of an example scenario manager associated with the system shown in FIG. 1, according to an aspect of the present disclosure.

FIG. 4 is a functional block diagram of an example scenario simulator associated with the system shown in FIG. 1, according to an aspect of the present disclosure.

FIG. 5 is a functional block diagram of an example analysis engine associated with the system shown in FIG. 1, according to an aspect of the present disclosure.

FIG. 6A is a functional block diagram of an example role-based planner associated with the system shown in FIG. 1, according to an aspect of the present disclosure.

FIG. 6B is an example illustrating example steps for generating a multi-stakeholder role/action plan associated with the role-based planner shown in FIG. 6A, according to an aspect of the present disclosure.

FIG. 7 is a flow chart diagram of an example method for generating an optimized action plan for multiple care stakeholders, associated with the system shown in FIG. 1, according to an aspect of the present disclosure.

FIG. 8 is a functional block diagram of an example computer system, according to an aspect of the present disclosure.

### DETAILED DESCRIPTION

Aspects of the present disclosure relate to systems and methods for creating an optimized electronic action plan for physician(s) and a care team with respect to healthcare process(s) for patient(s). An optimized action planner system of the present disclosure may simulate and generate an electronic recommended action plan for at least one physician and a care team, optimized for one or more outcomes. An electronic optimized action plan of the present disclosure may help the care team simulate and identify various outcomes based on one or more plans or workflow changes. The electronic optimized action plan may also help to identify driving factors for outcome, how changes to those factors may affect the outcome and by how much the changes may affect the outcome. The electronic optimized action plan may also allow the care team to define various cohorts of patients, and how diagnoses, care plan and severity of a condition may affect their probability of achieving a given outcome. The electronic optimized action plan may also provide incremental updates to recommendations based on each new patient, and over a period of time, in order to suitably adjust various factors towards achieving outcomes.

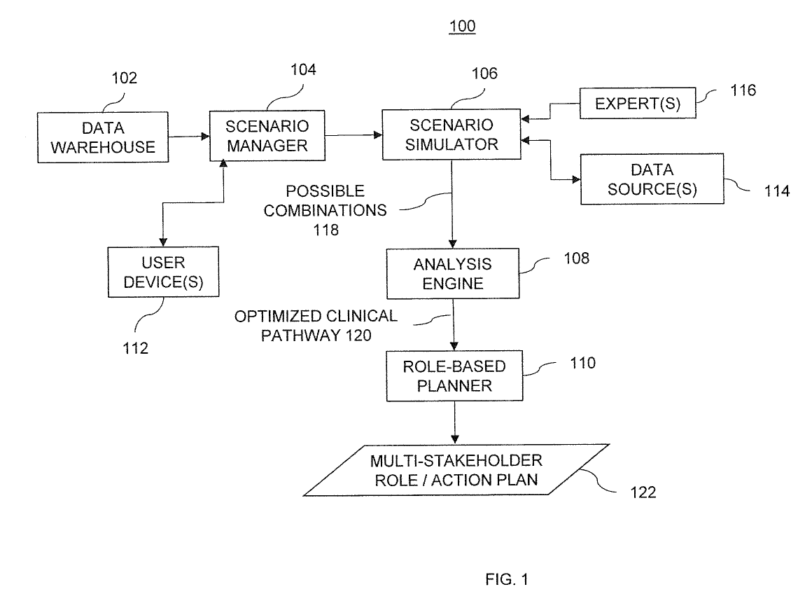

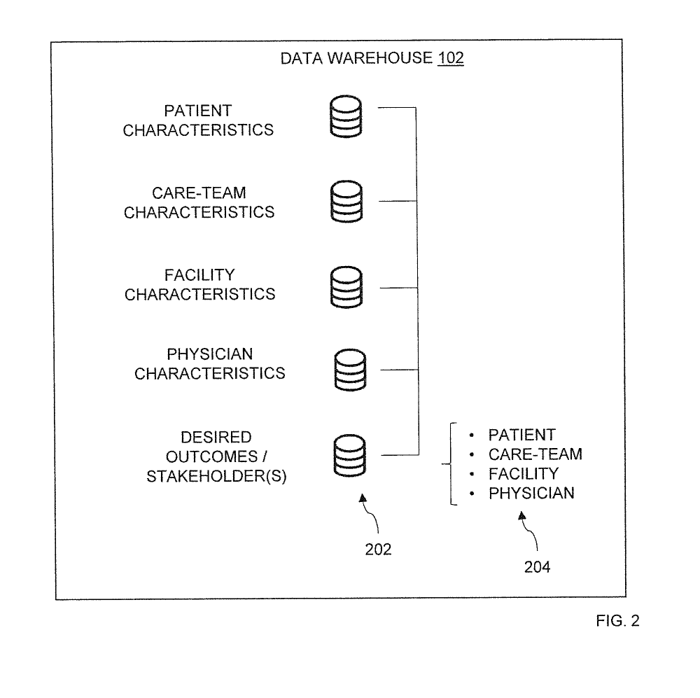

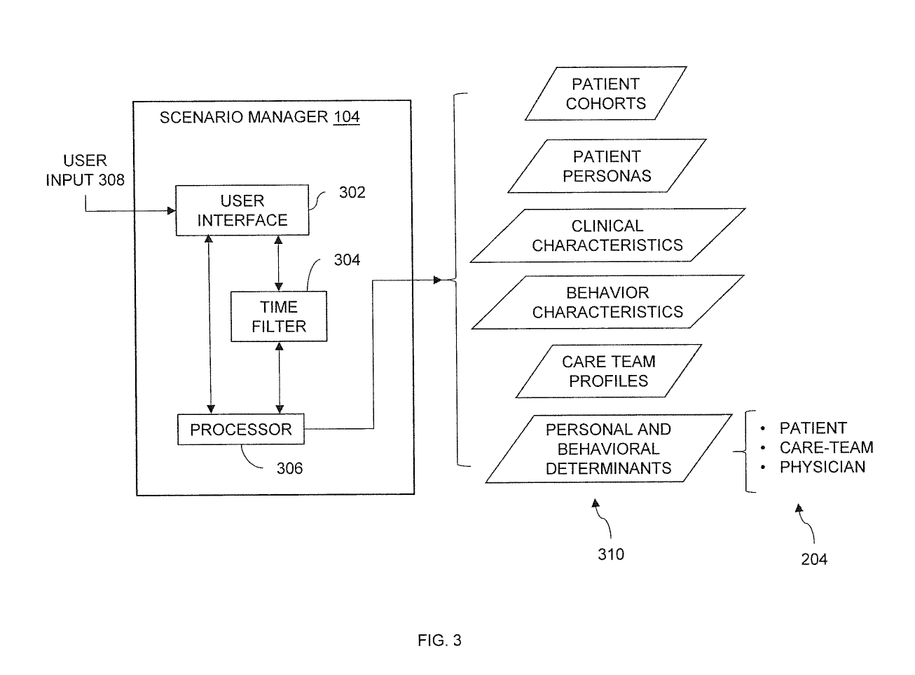

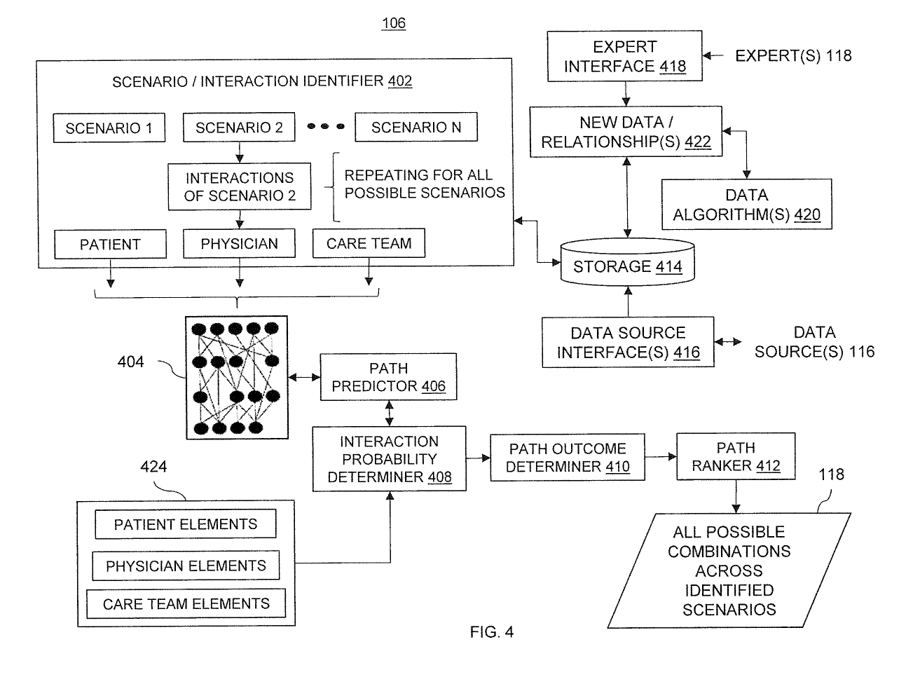

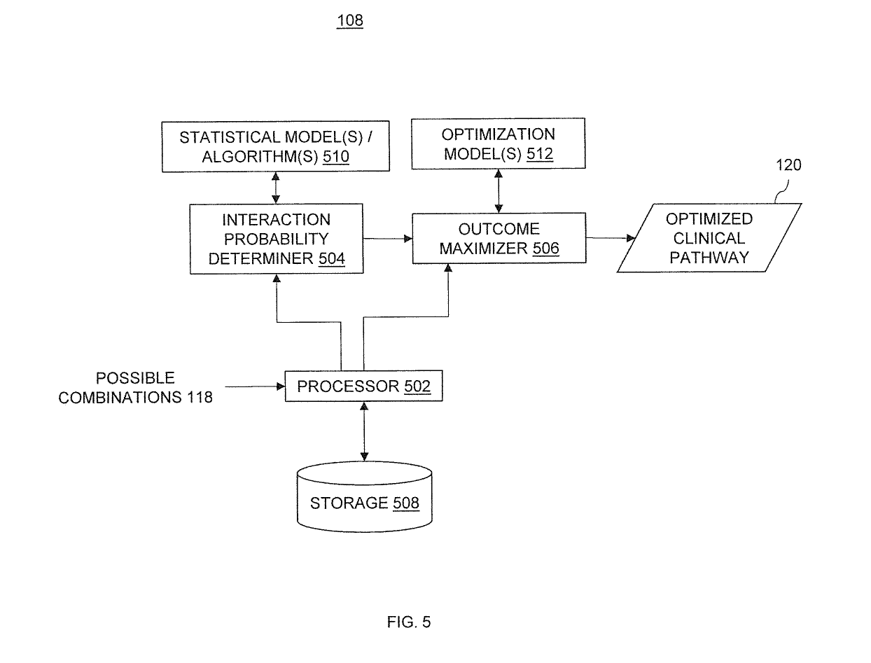

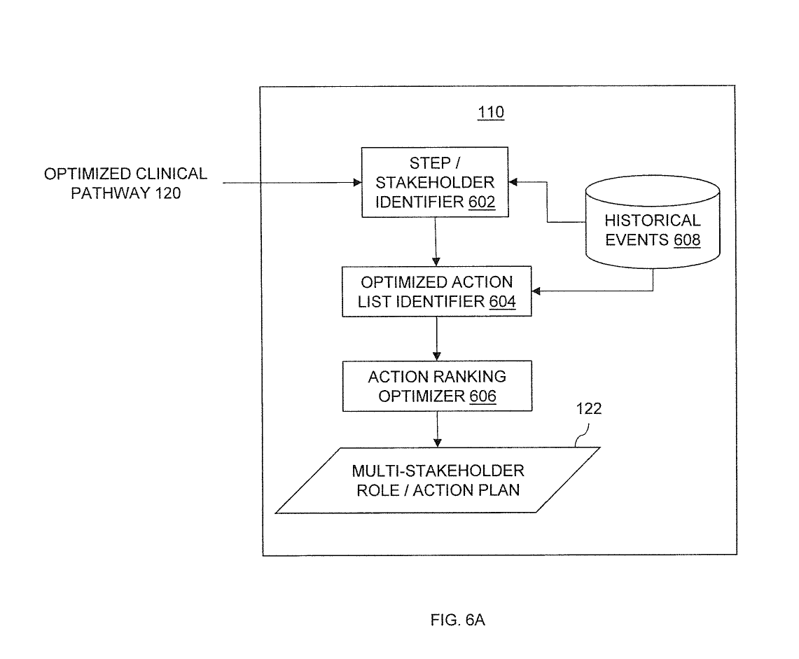

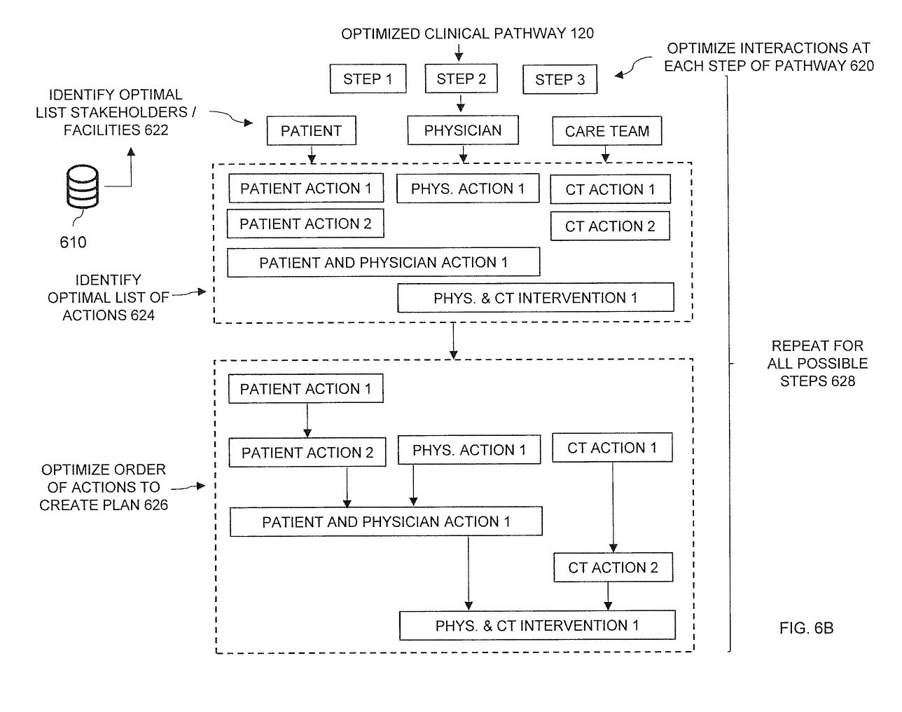

Referring to FIGS. 1-6B, optimized action planner system 100 (also referred to herein as system 100) is described, according to aspects of the present disclosure. In particular, FIG. 1 is a functional block diagram illustrating example optimized action planner system 100; FIG. 2 is a functional block diagram of example data warehouse 102 of system 100; FIG. 3 is a functional block diagram of example scenario manager 104 of system 100; FIG. 4 is a functional block diagram of example scenario simulator 106 of system 100; FIG. 5 is a functional block diagram of example analysis engine 108 of system 100; FIG. 6A is a functional block diagram of example role-based planner 110 of system 100; and FIG. 6B is an example illustrating example steps for generating a multi-stakeholder role/action plan 122 associated with role-based planner 110.

As shown in FIG. 1, system 100, in some examples, may comprise an outcome-based care simulation and planning system. System 100 may include data warehouse 102, scenario manager 104, scenario simulator 106, analysis engine 108 and role-based planner 110. System 100 may communicate with one or more user device(s) 112, for example, via user interface 302 (FIG. 3). For example, user device(s) 112 may communicate with one or more components of system 100 (e.g., data warehouse 102, scenario manager 104, scenario simulator 106, analysis engine 108 and/or role-based planner 110) via user interface 302. As another example, user device(s) 112 may directly communicate with one or more components of system 100 (e.g., data warehouse 102, scenario manager 104, scenario simulator 106, analysis engine 108 and/or role-based planner 110).

In some examples, system 100 may communicate with and obtain data from one or more data source(s) 114. In some examples, scenario simulator 106 may be configured to interact with one or more expert(s) 116, described further below with respect to FIG. 4.

Each of data warehouse 102, scenario manager 104, scenario simulator 106, analysis engine 108, role-based planner 110 and user device(s) 112 may comprise one or more computing devices, including a non-transitory memory storing computer-readable instructions executable by a processing device to perform the functions described herein. It should be understood that optimized action planner system 100 refers to a computing system having sufficient processing and memory capabilities to perform the specialized functions described herein.

Although not shown, system 100 may include a controller specially configured to control operation of data warehouse 102, scenario manager 104, scenario simulator 106, analysis engine 108 and/or role-based planner 110. The controller may include, for example, a processor, a microcontroller, a circuit, software and/or other hardware component(s).

In some examples, components of optimized action planner system 100 (e.g., data warehouse 102, scenario manager 104, scenario simulator 106, analysis engine 108 and role-based planner 110) may be embodied on a single computing device. In other examples, optimized action planner system 100 may refer to two or more computing devices distributed over several physical locations, connected by one or more wired and/or wireless links.

Data warehouse 102, scenario manager 104, scenario simulator 106, analysis engine 108, role-based planner 110, user device(s) 112 and data source(s) 114 may be communicatively coupled via one or more networks (not shown). The one or more networks may include, for example, a private network (e.g., a local area network (LAN), a wide area network (WAN), intranet, etc.) and/or a public network (e.g., the Internet).

User device(s) 112 may comprise a desktop computer, a laptop, a smartphone, tablet, or any other user device known in the art. A user may interact with user device(s) 112, for example, via a graphical user interface displayed on any type of display device including a computer monitor, a smart-phone screen, tablet, a laptop screen or any other device providing information to a user. User device(s) 112 may include any suitable user interface, user input component(s), output component(s), and communication component(s) for creation, transmission and receipt of electronic information and data related to data entry, data manipulation and data/information output (such as electronic multi-stakeholder role/action plan 122, also referred to herein as electronic MSRA plan 122). Users of system 100 may include, without being limited to, patients, care teams, physicians, subject matter experts and/or facility personnel.

Referring to FIG. 2, data warehouse 102 may include one or more databases 202 for storing various data/information from users of system 100. Data warehouse 102, in general, may store all metadata for available stakeholders 204 (e.g., patients, care team, facility, physicians). For example, data warehouse 102 may store patient characteristics, care team characteristics, facility characteristics, physician characteristics and desired outcomes/stakeholder information for each stakeholder 204. As shown in FIG. 1, data/information stored in data warehouse 102 may be provided to scenario manager 104.

Referring to FIG. 3, scenario manager 104 may include user interface 302, time filter 304 and processor 306. In some examples, scenario manager 104 may include storage (not shown). In general, scenario manager 104 may be configured to receive input 308 from one or more designated users (e.g., stakeholders 204) via user interface 302. The input(s) 308 may define data or parameters about one or more stakeholders 204. In some examples, processor 306 may be configured to control operation of one or more of user interface 302 and time filter 304. Processor 306 may also be configured to communicate with data warehouse 102, scenario simulator 106, analysis engine 108, role-based planner 110 and/or user device(s) 112 (e.g., via an interface, not shown).

Processor 306 may be configured to generate one or more categories of information 310 based on the received user input(s) 308. In some examples, processor 306 may be configured to store categories of information 310. In some examples, processor 306 may also be configured to receive electronic MSRA plan 122 (e.g., via role-based planner 110) and may cause user interface 302 to display at least a portion of electronic MSRA plan 122. In some examples, processor 306 may cause user interface 302 to present information and/or prompts for information associated with a particular stakeholder 204, so that different types of information/prompts may be displayed depending up the type of stakeholder 204. Processor 306 may include, without being limited to, a microprocessor, a central processing unit, an application specific integrated circuit (ASIC), a field programmable gate array (FPGA), a digital signal processor (DSP) and/or a network processor. In some examples, processor 306 may be configured with specially programmed processing logic that may cause processor 306 to execute the functions described herein.

User interface 302 may be configured to provide one or more prompts for information (e.g., data and/or parameters) regarding various stakeholders 204. In some examples, the prompts for information may vary dependent upon the type of designated user (e.g., whether the designated user is a patient, a physician, a care team individual, facility personnel, etc.) Scenario manager 104 may also include time filter 304, to assist in applying the received information to appropriate categories of information.

In general, user interface 302 may be configured to receive data and information from various designated stakeholders 204 (e.g., patients, care teams, physicians, facility personnel) for entry and/or manipulation by various components of system 100. User interface 302 may be configured to receive information from designated users (e.g., stakeholders 204) such that scenario manager 104, via processor 306, may define categories of information 310 including, without being limited to, one or more patient cohorts, patient personas, other clinical characteristics, behavioral characteristics, care team profiles, distribution of cases across diagnoses, social and personal determinants for patients, care team and physicians over a defined period of time (e.g., based on time filter 304), coping skills and social/family support, as well as any other actionable factors that may influence one or more specifically defined outcomes. In some examples, user interface 302 may also provide options for prompting a designated user to define one or more specific outcomes. Scenario manager 104, via processor 306, may determine the categories of information 310 based on the received inputs as well as based on data/information from data warehouse 102.

User interface 302 may also be configured to display results determined by system 100, including, for example, electronic MSRA plan 122. Information obtained by scenario manager 104 (e.g., via user interface 302) may also be stored in data warehouse 102.

In some examples, user interface 302 may be configured to generate a specialized graphical user interface (GUI) for the presentation, prompts, input, manipulation and/or selection of data/information 308 in one or more windows of a display screen (not shown) of user interface 302. In some examples, user interface 302 may include a software application having specially programmed instructions configured to render the GUI.

Referring to FIG. 4, scenario simulator 106 may be configured to receive the categories of information 310 (FIG. 3) defined by scenario manager 104 (as well as any other suitable data/information from data warehouse 102), and identify all possible scenarios as well as all possible combinations of interactions between stakeholders for each possible scenario. Scenario simulator 106 may further be configured to subsequently simulate the combinations of interactions for each possible scenario, to define potential outcomes as well as a quality of those outcomes.

Scenario simulator 106 may include scenario/interaction identifier 402, path simulator 404, path predictor 406, interaction probability determiner 408, path outcome determiner 410 and path ranker 412. In some examples, scenario simulator 106 may include storage 414. In some examples, scenario simulator 106 may also include one or more of at least one data source interface 416, at least one expert interface 418 and one or more data algorithms 420. In some examples, scenario simulator 106 may include a controller specially configured to control operation of one or more of components 402-424. In some examples, scenario simulator 106 may include, for example, a processor, a microcontroller, a circuit, software and/or other hardware component(s).

Scenario/interaction identifier 402 may be configured to identify all possible scenarios (e.g., scenario 1, scenario 2, . . . , scenario N, where N is a non-negative integer greater than or equal to 1). Scenario/interaction identifier 402 may also be configured to identify all possible combinations of interactions between stakeholders 204 (e.g., a patient, a physician, a care team) for each possible scenario (such as interactions of scenario 2, as illustrated in FIG. 4). Scenario/interaction identifier 402 may consider each entity, including institution, care team, physician, patient, caregiver/family etc. In some examples, all relationships and interactions between entities may be defined and updated with new data and as new relationships 422 are discovered.

In some examples, scenario simulator 108 may automatically discover new data/relationships 422 (e.g., via one or more data analysis algorithm(s) 420). In some examples, scenario simulator 108 may receive indications of new data/relationships 422 from expert(s) 118 via expert interface 418.

In some examples, scenario simulator 108 may include data source interface(s) 416. Data source interface(s) 416 may be configured to communicate with data source(s) 116, for example, to obtain data on stakeholder relationships and/or interactions from among data source(s) 116. Data source(s) 116 may include any suitable source of data for obtaining stakeholder relationships and/or interactions (e.g., interactions between patients and various physicians). For example, data source(s) 116 may include, without being limited to, electronic medical data systems, behavioral data systems, invasive or non-invasive wearable and/or monitoring devices, electronic databases associated with one or more of an insurance organization, a hospital, a physician medical practice, an outpatient clinic and an urgent care facility, social media, news sources, etc. The obtained data may be stored in storage 414.

Storage 414 may include any suitable non-transitory computer readable storage medium for receiving, storing and retrieving electronic data. Storage 414 may include, without being limited to, at least one of a database, a read-only memory (ROM), a random access memory (RAM), a flash memory, a dynamic RAM (DRAM) and a static RAM (SRAM).

In some examples, scenario simulator 106 may include expert interface(s) 418. Expert interface(s) 418 may be configured for interaction with expert(s) 118 (for example, data scientist(s), subject matter experts, etc.). Expert interface(s) 422 may be configured to provide at least a portion of the data stored in storage 414 for review and/or analysis by expert(s) 118. Expert interface(s) 418 may be configured to receive indications of new data, new relationships and/or interactions 422 (also referred to herein as new data 422) from expert(s) 118. In some examples, new data 422 may be stored in storage 414. In some examples, expert interface(s) 418 may be configured to provide a user interface, such as a GUI, for interaction with expert(s) 118. In some examples, expert interface 418 may be configured to present and/or allow manipulation of different information depending on the type of expert interacting with scenario simulator 106.

In some examples, scenario simulator 106 may include one or more data analysis algorithms 420 for automatically discovering new data 422 from among data stored in storage 414. Data analysis algorithm(s) 420 may include any suitable algorithm for data mining and/or classification, including but not limited to machine learning, statistical algorithms, neural networks, artificial intelligence, etc.

Path simulator 404 may receive the defined and updated relationships and interactions from scenario/interaction identifier 402. Based on the received data from scenario/interaction identifier 402, path simulator 404 may be configured to simulate all possible paths for each given scenario (e.g., scenario 1, scenario 2, . . . , scenario N). Path simulator 404 may send the simulated possible paths to path predictor 406.

Path predictor 406 may receive the simulated possible paths from path simulator 404, and may be configured to predict one or more most likely paths. Path predicator 406 may be configured to predict the most likely paths based on, for example, at least one of one or more predetermined rules, one or more machine learning models and other artificial intelligence (AI) models trained on historical data. Path predictor 406 may send the predicted most likely path(s) to interaction probability determiner 408.

Interaction probability determiner 408 may be configured to receive the predicted most likely path(s) form path predictor 406. Responsive to the predicted most likely path(s), interaction probability determiner 408 may be configured to determine and/or update a probability for each interaction. The probability for interaction may also be based on data available for each data element 424 (e.g., one or more patient elements, one or more physician elements, one or more facility elements, one or more care team elements).

In some examples, scenario simulator 106 may be configured to scrape and source data about one or more entities, longitudinally or episodic, in near real-time or via batch mode data, either automatically or via manual upload or database connection (e.g., via data source(s) 116). Interaction probability determiner 408, in some examples, may use this additional data to determine the probabilities for interaction (for each interaction). Interaction probability determiner 408 may send the determined probabilities for interaction to path outcome determiner 410.

Path outcome determiner 410 may receive three determined probabilities for interaction from interaction probability determiner 408. Based on the determined probability(s), Path outcome determiner 410 may calculate and/or update the outcome for each path (based on the data). Path outcome determiner 410 may also be configured to determine a quality of outcome of each path. For example, a quality of outcome for combination 1 in Scenario 2 may be given a quality of either high, medium or low (e.g., by comparing the probabilities for interaction to one or more predetermined thresholds). Path outcome determiner 410 may send the determined path outcomes and any quality determinations to path ranker 412.

Path ranker 412 may receive the determined path outcomes from path outcome determiner 410, and may be configured to rank order the paths. Path ranker 412 may be configured to rank order the paths to optimize and/or maximize a given outcome (as defined in scenario manager 106). Accordingly, the best paths may be rank ordered by path ranker 412 and may be provided to analysis engine 108 (FIG. 1) for review (as part of output data 118). In particular, path ranker 412 may generate output data 118 including all possible combinations of paths as ranked, across all identified scenarios (e.g., scenario 1, scenario 2, . . . , scenario N), including, in some examples, any outcome quality information.

Scenario simulator 106 may also access relevant reference relationships and other models (e.g., stored in storage 414, data warehouse 102, from data source(s) 114) that may inform and enhance each step of the simulated paths as well as local and overall accuracies for each path and outcome optimization. Non-limiting examples of reference relationships/models may include illness beliefs, cognitive models, common sense reasoning models, emotion and other psychosocial models of human cognition, emotion and decision making. Reference relationships/models may also include, for example, models of irrational human decision making, based on training machine learning models and AI models on such actions and decisions of entities in the past.

Referring to FIG. 5, analysis engine 108 may include processor 502, interaction probability determiner 504, outcome maximizer 506, storage 508, statistical model(s)/algorithm(s) 510 and one or more optimization models 512. Processor 502 may be configured to control one or more of interaction probability determiner 504, outcome maximizer 506, storage 508, statistical model(s)/algorithm(s) 510 and optimization model 512.

Storage 508 may be configured to store historical data on previous interactions between stakeholders 204. Storage 508 may also be configured to store a list and data values on all data elements used to define each entity and relationships and interactions. Storage 508 may also be configured to store various factors that may be directly or indirectly relevant to each scenario. In general, data/information stored in storage 508 may be used to by one or more components of analysis engine 108 to determine optimized clinical pathway 120. Storage 508 may include any suitable non-transitory computer readable storage medium for receiving, storing and retrieving electronic data. Storage 508 may include, without being limited to, at least one of a database, ROM, RAM, flash memory, DRAM and SRAM.

Processor 502 may be configured to receive possible combinations 118 (of all possible identified scenarios), from scenario simulator 106, and to obtain historical data on previous interactions between stakeholders 204 (e.g., from storage 508) as inputs. Based on possible combinations 118 and the historical data, processor 502 may control operation of interaction probability determiner 504 (in combination with one or more statistical models of one or more advanced statistical algorithms 510, also referred to herein as statistical model(s)/algorithm(s) 510) and outcome maximizer 506 (in combination with one or more optimization models 512) to compute, optimize and identify a best (i.e., optimized) clinical pathway 120. Processor 502 (or outcome maximizer 506) may be configured to provide optimized clinical pathway 120 to role-based planner 110 (FIG. 1). Processor 502 may include, without being limited to, a microprocessor, a central processing unit, an ASIC, an FPGA, a DSP and/or a network processor.

Interaction probability determiner 504 may be configured to use possible combinations 118 and historical data to train and apply statistical models of algorithms(s) 510. Interaction probability determiner 504 may also be configured to determine probabilities for each interaction and a probability for an overall interaction based on (trained) statistical model(s)/algorithm(s) 510. Based on the computed probabilities for each possible path, as well as raw data on each data element (e.g., stored in storage 508), interaction probability determiner 504 may be configured to use algorithm(s) 510 (e.g., one or more statistical, machine learning and/or AI algorithms) to identify key drivers of outcomes. Interaction probability determiner 504 may also be configured to identify a level of influence of each driver on the outcome.

Outcome maximizer 506 may be configured to develop and run one or more optimization models 512 to maximize the outcome, based on the probability for an overall interaction (determined by interaction probability determiner 504). Outcome maximizer 506 may be configured to determine optimized clinical pathway 120, based on the optimized outcome. As part of the optimization, outcome maximizer 506 may identify combinations and/or sequence(s) of data elements (e.g., in storage 508) that may have a higher impact on outcomes. Analysis engine 108 may provide the optimized clinical pathway to role-based planner 110 (FIG. 1).

Referring to FIGS. 6A and 6B, example role-based planner 110 is described (FIG. 6A) and illustrated with respect to an example (FIG. 6B). As shown in FIG. 6A, role-based planner 110 may include step/stakeholder identifier 602, optimized action list identifier 604, action ranking optimizer 606 and storage 608. In some examples, role-based planner 110 may include a controller specially configured to control operation of one or more of components 602-608. In some examples, role-based planner 110 may include, for example, a processor, a microcontroller, a circuit, software and/or other hardware component(s).

Storage 608 may be configured to store reference knowledge (e.g., data) associated with one or more historical events (described further below). Storage 608 may be configured to store any suitable data/information for determining electronic MSRA plan 122. Storage 608 may include any suitable non-transitory computer readable storage medium for receiving, storing and retrieving electronic data. Storage 508 may include, without being limited to, at least one of a database, ROM, RAM, flash memory, DRAM and SRAM.

Step/stakeholder identifier 602 of role-based planner 110 may be configured to receive optimized clinical pathway 120 (from analysis engine 108) and utilize optimized clinical pathway 120 to identify all possible steps for all included stakeholders. Step/stakeholder 602 may be configured to obtain reference knowledge of relevant historical events from storage 608, and identify an optimal list of one or more stakeholders and, in some examples, one or more facilities for each step of optimized clinical pathway 120. For example, as shown in FIG. 6B, optimized clinical pathway 120 may be used, by step/stakeholder identifier 602, to identify three steps (step 1, step 2, step 3), and optimize interactions at each step of the pathway (620). Step/stakeholder identifier 602 may also identify an optimal list of stakeholders/facilities (622). For example, in FIG. 6B, the optimal list of stakeholders includes a patient, a physician and a care team.

Referring to FIGS. 6A and 6B, optimized action list identifier 604 may be configured to identify an optimal list of desired actions (624), including, in some examples, based on reference knowledge of historical events stored in storage 608. For example, for step 2, two patient actions are identified (patient action 1, patient action 2), one physician action is identified (phys. action 1), two care team (CT) actions are identified (CT action 1, CT action 2), one combined patient and physician action is identified (patient and physician action 1) and one combined physician and care team intervention is identified (Phys. & CT intervention 1).

Action ranking optimizer 606 may be configured to assign an order to all desired actions and owners for all the desired actions (626), based on the optimal list of actions. For example, as shown in FIG. 6B, for step 2, action ranking optimizer 606 has ordered patient action 1 as occurring first, a set of patient action 2, physician action 1 and care team action 1 as occurring second, combined patient and physician action 1 as occurring third, care team action 2 as occurring fourth and combined physician and care team intervention 1 as occurring fifth.

As shown in FIG. 6B, the same identification of an optimal list of actions (624) and optimized order of actions (626) may be repeated (628) for all possible steps (e.g., step 1 and step 3) of optimized clinical pathway 120. The optimized order of actions over all steps of optimized clinical pathway 120 may be used to generate electronic MSRA plan 122 for each identified stakeholder/facility.

In operation, role-based planner 110 may prioritize and rank orders into a best (optimized) configuration of actions/interventions, based on factors identified analysis engine 108 (FIG. 1), including optimized clinical pathway 120. Role-based planner 110 may identify a most suitable role/person(s) for each action/intervention, based on a reference knowledgebase (e.g., historical events in storage 608) and a predicted likelihood of their actions and success probability. In some examples, role-based planner 110 may, accordingly, arrange the actions under two or more configurations: for example, one configuration as an overall plan, and the other configuration as a plan for each role. One or more role-specific preferences, such as availability, cohort age preference etc. could be included as preferential filters based on which the plans could be refined and created. Together, the two configurations may form electronic MSRA plan 122.

In some examples, role-based planner 110 may be connected to one or more sources of data, and may be incrementally updated based on changes in data.

Referring to FIG. 1, role(s) defined in system 100 may include one or more human or institutional entities. A care team, in some examples, may also include family members, friends and other closely relevant individuals, directly or indirectly related. A recommendation may be performed for one or more clinical and/or behavioral conditions and/or specialties. Factors that may influence outcomes may be based, in some examples, on data collected in the past, data collected in real-time or predicted and validated for events and interactions in a near future. Institutions may include, without being limited to, hospitals, clinics, health systems, insurers, affinity groups and associations, employers and/or government bodies.

In some examples, system 100 may be configurable to function across various language-specific roles, international roles and related cultural and behavioral factors, personalization and psychosocial beliefs, attitudes and sensitivities.

Some portions of above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. The described operations and their associated components may be embodied in specialized software, firmware, specially-configured hardware or any combinations thereof.

It may be appreciated that the operations shown in FIGS. 1-6B may be performed by processing logic that may comprise hardware (e.g., circuitry, dedicated logic, programmable logic, microcode, etc.), software (such as instructions run on a processing device), or a combination thereof.

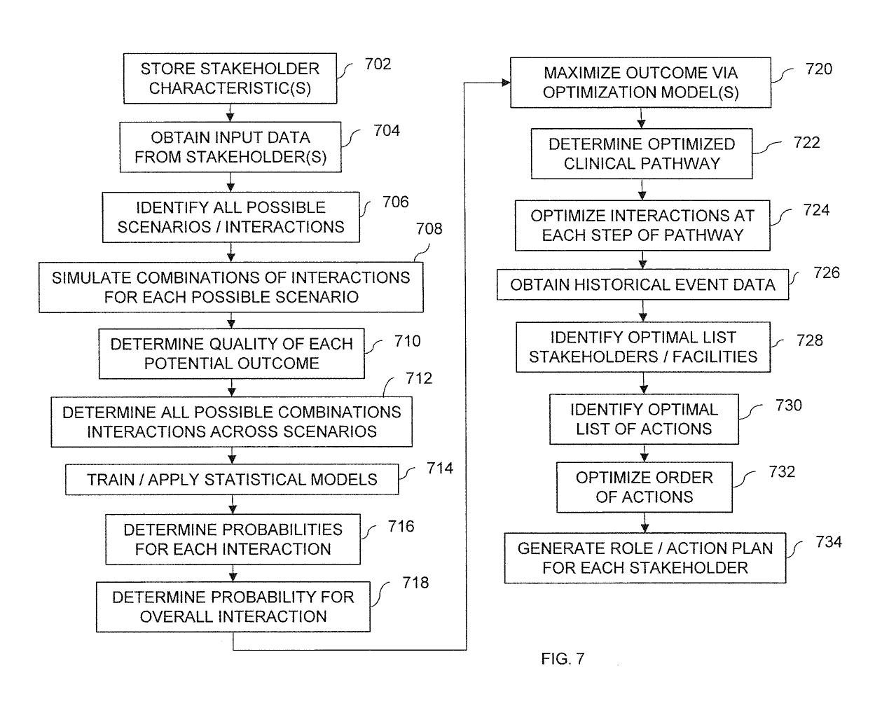

As illustrated in FIG. 7, the method shown may be performed by processing logic that may comprise hardware (e.g., circuitry, dedicated logic, programmable logic, microcode, etc.), software (such as instructions run on a processing device), or a combination thereof. In one embodiment, the method shown in FIG. 7 may be performed by one or more specialized processing components associated with components of optimized action planner system 100 of FIGS. 1-6B. FIG. 7 is described with respect to FIGS. 1-6B.

FIG. 7 illustrate a method for generating an optimized action plan for multiple stakeholders, via multi-faceted optimized action planner system 100. At step 702, characteristics 202 for various stakeholders 204 may be stored in data warehouse 102. At step 704, scenario manager 104 may prompt and receive inputs 308 from one or more designated users, via user interface 302, with respect to relevant data about care stakeholders including, for example, patients, physicians, care-teams as well as facilities. At step 704, scenario manager 104 may also determine various categories of information 310 for the various stakeholders 204 based on the received inputs 308 from the designated user(s).

At step 706, scenario simulator 106 may identify all possible scenarios for each stakeholder 204 (e.g., patient, physician, care team) as well as all possible combinations of interactions between stakeholders, based at least in part on the categories of information (determined in step 704). At step 708, scenario simulator 106 may simulate the combinations of interactions for each possible scenario. At step 710, scenario simulator 106 may determine a quality of each potential outcome. At step 712, scenario simulator 106 may determine all possible combinations of interactions between stakeholders across the identified scenarios (possible combinations 118), and may transmit the set of possible combinations 118 along with the quality of outcomes to analysis engine 108.

At steps 714-722, the sets of possible combinations (determined at step 714) may be used by analysis engine 108, along with case specific and historical case data, to determine optimized clinical pathway 120 for all stakeholders. More specifically, at step 714, the set of possible combinations 118 and historical data may be used to train and apply statistical models of algorithms(s) 510. At step 716, analysis engine 108 may determine probabilities for each interaction. At step 718, analysis engine 108 may determine a probability for an overall interaction. At step 720, analysis engine 108 may develop and run optimization model(s) 512 for maximizing the outcome. At step 722, analysis engine 108 may then determine optimized clinical pathway 120.

Optimized clinical pathway 120 (determined at step 722) may be fed into role-based planner 110, and role-based planner 110 may identify an order of all desired actions as well as the stakeholder needed to perform those actions in order to achieve the desired outcome (steps 724-734). More specifically, at step 724, role-based planner 110 may optimize interactions at each step of the optimized clinical pathway. At step 726, role-based planner 110 may obtain reference knowledge of relevant historical events (e.g., via storage 608). At step 728, role-based planner 110 may identify an optimal list of stakeholders and facilities, based at least in part on the obtained reference knowledge, for each step of optimized clinical pathway 120. At step 730, role-based planner 110 may identify an optimal list of desired actions for each step of optimized clinical pathway 120. At step 732, role-based planner 110 may optimize the order of actions. At step 734, role-based planner 110 may generate electronic MSRA plan 122 for each stakeholder based on the optimized order of actions (determined in step 732).

In some examples, at least a portion of MSRA plan 122 may be transmitted to the identified stakeholders for performance of the actions/roles assigned by MSRA plan 122. In some examples, scenario manager 104 may manage transmission and presentation of MSRA plan 122 to the identified stakeholders. In some examples, scenario manager 104 may further prompt the identified stakeholders for additional information and/or provide alerts to the identified stakeholders, to confirm that the identified stakeholders follow MSRA plan 122. In some examples, scenario manager 104 may update one or more of data warehouse 102, scenario simulator 106, analysis engine 108 and role-based planner 110 with updated information obtained by one or more of the identified stakeholders. In some examples, system 100 may modify MSRA plan 122 based on updated information from among the identified stakeholders.

Systems and methods of the present disclosure may include and/or may be implemented by one or more specialized computers including specialized hardware and/or software components. For purposes of this disclosure, a specialized computer may be a programmable machine capable of performing arithmetic and/or logical operations and specially programmed to perform the functions described herein. In some embodiments, computers may comprise processors, memories, data storage devices, and/or other commonly known or novel components. These components may be connected physically or through network or wireless links. Computers may also comprise software which may direct the operations of the aforementioned components. Computers may be referred to with terms that are commonly used by those of ordinary skill in the relevant arts, such as servers, personal computers (PCs), mobile devices, and other terms. It will be understood by those of ordinary skill that those terms used herein are interchangeable, and any special purpose computer capable of performing the described functions may be used.

Computers may be linked to one another via one or more networks. A network may be any plurality of completely or partially interconnected computers wherein some or all of the computers are able to communicate with one another. It will be understood by those of ordinary skill that connections between computers may be wired in some cases (e.g., via wired TCP connection or other wired connection) or may be wireless (e.g., via a WiFi network connection). Any connection through which at least two computers may exchange data can be the basis of a network. Furthermore, separate networks may be able to be interconnected such that one or more computers within one network may communicate with one or more computers in another network. In such a case, the plurality of separate networks may optionally be considered to be a single network.

The term “computer” shall refer to any electronic device or devices, including those having capabilities to be utilized in connection with optimized action planner system 100 (including components 102-114), such as any device capable of receiving, transmitting, processing and/or using data and information. The computer may comprise a server, a processor, a microprocessor, a personal computer, such as a laptop, palm PC, desktop or workstation, a network server, a mainframe, an electronic wired or wireless device, such as for example, a telephone, a cellular telephone, a personal digital assistant, a smartphone, an interactive television, such as for example, a television adapted to be connected to the Internet or an electronic device adapted for use with a television, an electronic pager or any other computing and/or communication device.

The term “network” shall refer to any type of network or networks, including those capable of being utilized in connection with the system described herein, such as, for example, any public and/or private networks, including, for instance, the Internet, an intranet, or an extranet, any wired or wireless networks or combinations thereof.

The term “computer-readable storage medium” should be taken to include a single medium or multiple media that store one or more sets of instructions. The term “computer-readable storage medium” shall also be taken to include any medium that is capable of storing or encoding a set of instructions for execution by the machine and that causes the machine to perform any one or more of the methodologies of the present disclosure.

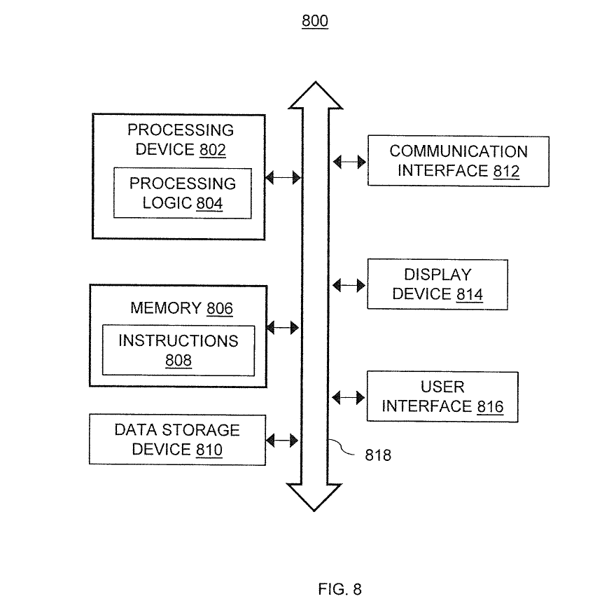

FIG. 8 illustrates a functional block diagram of a machine in the example form of computer system 800 within which a set of instructions for causing the machine to perform any one or more of the methodologies, processes or functions discussed herein may be executed. In some examples, the machine may be connected (e.g., networked) to other machines as described above. The machine may operate in the capacity of a server or a client machine in a client-server network environment, or as a peer machine in a peer-to-peer (or distributed) network environment. The machine may be any special-purpose machine capable of executing a set of instructions (sequential or otherwise) that specify actions to be taken by that machine for performing the functions describe herein, Further, while only a single machine is illustrated, the term “machine” shall also be taken to include any collection of machines that individually or jointly execute a set (or multiple sets) of instructions to perform any one or more of the methodologies discussed herein. In some examples, one or more components of optimized action planner system 100 (data warehouse 102, scenario manager 104, scenario simulator 106, analysis engine 108, role-based planner 110, user device(s) 112 and/or data source(s) 114) may be implemented by the example machine shown in FIG. 8 (or a combination of two or more of such machines).

Example computer system 800 may include processing device 802, memory 806, data storage device 810 and communication interface 812, which may communicate with each other via data and control bus 818. In some examples, computer system 800 may also include display device 814 and/or user interface 816.

Processing device 802 may include, without being limited to, a microprocessor, a central processing unit, an ASIC, a FPGA, a DSP and/or a network processor. Processing device 802 may be configured to execute processing logic 804 for performing the operations described herein. In general, processing device 802 may include any suitable special-purpose processing device specially programmed with processing logic 804 to perform the operations described herein.

Memory 806 may include, for example, without being limited to, at least one of a read-only memory (ROM), a RAM, a flash memory, a DRAM and a SRAM, storing computer-readable instructions 808 executable by processing device 802. In general, memory 806 may include any suitable non-transitory computer readable storage medium storing computer-readable instructions 808 executable by processing device 802 for performing the operations described herein. For example, computer-readable instructions 808 may include operations performed by components 102-112 of optimized action planner system 100), including operations shown in FIG. 7). Although one memory device 806 is illustrated in FIG. 8, in some examples, computer system 800 may include two or more memory devices (e.g., dynamic memory and static memory).

Computer system 800 may include communication interface device 812, for direct communication with other computers (including wired and/or wireless communication) and/or for communication with a network. In some examples, computer system 800 may include display device 814 (e.g., a liquid crystal display (LCD), a touch sensitive display, etc.). In some examples, computer system 800 may include user interface 816 (e.g., an alphanumeric input device, a cursor control device, etc.).

In some examples, computer system 800 may include data storage device 810 storing instructions (e.g., software) for performing any one or more of the functions described herein. Data storage device 810 may include any suitable non-transitory computer-readable storage medium, including, without being limited to, solid-state memories, optical media and magnetic media.

While the present disclosure has been discussed in terms of certain embodiments, it should be appreciated that the present disclosure is not so limited. The embodiments are explained herein by way of example, and there are numerous modifications, variations and other embodiments that may be employed that would still be within the scope of the present disclosure.

1. An optimized action planner system comprising:
a user interface configured to receive data comprising at least one of characteristics of a patient, a physician and one or more other treatment personnel defining a care team;
a scenario manager configured to organize the received data into multiple categories of information;
a scenario simulator configured to:
identify a plurality of possible scenarios and possible combinations of interactions between the patient, the physician and the care team with a respect to a predefined healthcare outcome, based at least in part on the multiple categories of information, and
define a set of potential outcomes among the plurality of possible scenarios and possible combinations via simulation according to one or more statistical algorithms, based on the predefined healthcare outcome;
an analysis engine configured to determine an optimized clinical pathway based on the set of potential outcomes, according to one or more further statistical algorithms; and
a role-based planner configured to determine an electronic action plan based on the optimized clinical pathway, the electronic action plan identifying one or more roles and one or more actions for each of the patient, the physician and the care team.

2. The optimized action planner system of claim 1, wherein the categories of information include at least one of patient cohorts, patient personas, clinical characteristics, behavioral characteristics, treatment team profiles, personal determinants and behavioral determinants.

3. The optimized action planner system of claim 1, wherein the user interface is configured to display at least a portion of the electronic action plan to one or more of the patient, the physician and the care team.

4. The optimized action planner system of claim 1, wherein the patient, the physician and the care team comprise users of different user categories, and the user interface is configured to prompt at least one of the users for information according to the different user categories, such that the received data corresponds to the prompted information.

5. The optimized action planner system of claim 1, wherein the scenario simulator is configured to simulate a plurality of possible clinical paths for each possible scenario, predict one or more likely clinical paths, determine one or more probable outcomes of each of the one or more predicted likely clinical paths, and define the set of potential outcomes based on the one or more probable outcomes.

6. The optimized action planner system of claim 5, wherein the scenario simulator is configured to rank the one or more predicted likely clinical paths based on the one or more probable outcomes.

7. The optimized action planner system of claim 1, where the scenario simulator is configured to define the set of potential outcomes based at least in part on reference data including at least one of predetermined illness beliefs, cognitive models, common sense reasoning models, psychosocial models of human cognition, emotion and decision making information and models of irrational human decision making.

8. The optimized action planner system of claim 1, wherein the scenario simulator is configured to identify new data including at least one of new relationship data and new interaction data from among one or more data sources, and the identification of the plurality of possible scenarios and possible interactions is based at least in part on the identified new data.

9. The optimized action planner system of claim 8, wherein the scenario simulator is configured to identify the new data based on one or more data algorithms.

10. The optimized action planner system of claim 8, wherein the scenario simulator further includes an expert interface configured to display at least a portion of data from among the one or more data sources, and to receive an indication identifying the new data based on the displayed portion of data.

11. The optimized action planner system of claim 1, wherein the analysis engine is configured to define the optimized clinical pathway based at least in part on historical interaction data and to identify the optimized clinical pathway according to optimization of the set of potential outcomes via at least one optimization model.

12. The optimized action planner system of claim 1, wherein the role-based planner is configured to rank and prioritize the one or more roles and the one or more actions for each of the patient, the physician and the care team.

13. The optimized action planner system of claim 1, wherein the role-based planner is configured to determine the electronic action plan based on at least one of historical event data, clinical condition data, behavioral condition data, specialty information, current event data and predicted event information.

14. The optimized action planner system of claim 1, further comprising a data warehouse configured to store at least one of patient characteristics, care team characteristics, facility characteristics, physician characteristics and one or more predefined outcomes.

15. The optimized action planner system of claim 1, wherein at least one of the one or more statistical algorithms and the one or more further statistical algorithms include one or more of machine learning, artificial intelligence and statistical processing techniques.

16. A method for creating an optimized action plan for multiple stakeholders, the method comprising:
receiving, via a user interface of an optimized action planner system, data comprising at least one of characteristics of a patient, a physician and one or more other treatment personnel defining a care team;
organizing, by a scenario manager of the optimized action planner system, the received data into multiple categories of information;
identifying, by a scenario simulator of the optimized action planner system, a plurality of possible scenarios and possible combinations of interactions between the patient, the physician and the care team with a respect to a predefined healthcare outcome, based at least in part on the multiple categories of information;
defining, by the scenario simulator, a set of potential outcomes among the plurality of possible scenarios and possible combinations via simulation according to one or more statistical algorithms, based on the predefined healthcare outcome;
defining, by an analysis engine of the optimized action planner system, an optimized clinical pathway based on the set of potential outcomes, according to one or more further statistical algorithms; and
determining, by a role-based planner of the optimized action planner system, an electronic action plan based on the optimized clinical pathway, the electronic action plan identifying one or more roles and one or more actions for each of the patient, the physician and the care team.

17. The method of claim 16, wherein the categories of information include at least one of patient cohorts, patient personas, clinical characteristics, behavioral characteristics, treatment team profiles, personal determinants and behavioral determinants.

18. The method of claim 16, the method further comprising displaying, via the user interface, at least a portion of the electronic action plan to one or more of the patient, the physician and the care team.

19. The method of claim 16, wherein the patient, the physician and the care team comprise users of different user categories, and the method further comprises prompting, by the user interface, at least one of the users for information according to the different user categories, such that the received data corresponds to the prompted information.

20. The method of claim 16, the method further comprising:
simulating, by the scenario simulator, a plurality of possible clinical paths for each possible scenario;
predicting, by the scenario simulator, one or more likely clinical paths based on the simulated plurality of possible clinical paths;
determining, by the scenario simulator, one or more probable outcomes of each of the one or more predicted likely clinical paths; and
defining, by the scenario simulator, the set of potential outcomes based on the one or more probable outcomes.

21. The method of claim 20, the method further comprising:
ranking, by the scenario simulator i the one or more predicted likely clinical paths based on the one or more probable outcomes.

22. The method of claim 16, wherein the defining of the set of potential outcomes includes defining the set of potential outcomes based at least in part on reference data including at least one of predetermined illness beliefs, cognitive models, common sense reasoning models, psychosocial models of human cognition, emotion and decision making information and models of irrational human decision making.

23. The method of claim 16, the method further comprising:
identifying, by the scenario simulator, new data including at least one of new relationship data and new interaction data from among one or more data sources; and
identifying, by the scenario simulator, the plurality of possible scenarios and possible interactions based at least in part on the identified new data.

24. The method of claim 23, wherein the identifying of the new data includes identifying the new data based on one or more data algorithms.

25. The method of claim 23, wherein the identifying of the new data includes:
displaying, via an expert interface, at least a portion of data from among the one or more data sources, and
receive an indication, via the expert interface, identifying the new data based on the displayed portion of data.

26. The method of claim 16, wherein the defining of the optimized clinical pathway includes:
defining the optimized clinical pathway based at least in part on historical interaction data, and
identifying the optimized clinical pathway according to optimization of the set of potential outcomes via at least one optimization model.

27. The method of claim 16, the method further comprising:
ranking and prioritizing, by the role-based planner, the one or more roles and the one or more actions for each of the patient, the physician and the care team.

28. The method of claim 16, wherein the determining of the electronic action plan includes determining the electronic action plan based on at least one of historical event data, clinical condition data, behavioral condition data, specialty information, current event data and predicted event information.

29. A non-transitory computer readable medium storing computer readable instructions that, when executed by one or more processing devices, cause the one or more processing devices to perform the functions comprising:
receiving, via a user interface, data comprising at least one of characteristics of a patient, a physician and one or more other treatment personnel defining a care team;
organizing the received data into multiple categories of information;
identifying a plurality of possible scenarios and possible combinations of interactions between the patient, the physician and the care team with a respect to a predefined healthcare outcome, based at least in part on the multiple categories of information;
defining a set of potential outcomes among the plurality of possible scenarios and possible combinations via simulation according to one or more statistical algorithms, based on the predefined healthcare outcome;
defining an optimized clinical pathway based on the set of potential outcomes, according to one or more further statistical algorithms; and
determining an electronic action plan based on the optimized clinical pathway, the electronic action plan identifying one or more roles and one or more actions for each of the patient, the physician and the care team.

30. The non-transitory computer readable medium of claim 29, wherein the categories of information include at least one of patient cohorts, patient personas, clinical characteristics, behavioral characteristics, treatment team profiles, personal determinants and behavioral determinants.
